(19) United States
(12) Patent Application Publication
(10) Pub. No.: US 2024/0353420 A1
(43) Pub. Date: Oct. 24, 2024

(54) NANOMATERIAL-ENHANCED CHIPS AND THEIR APPLICATIONS IN LASER DESORPTION IONIZATION MASS SPECTROMETRY DETECTION OF SMALL MOLECULE METABOLITES

(71) Applicant: Shanghai Jiao Tong University, Shanghai (CN)

(72) Inventors: Kun QIAN, Shanghai (CN); Jing CAO, Shanghai (CN); Shunxiang LI, Shanghai (CN)

(73) Assignee: Shanghai Jiao Tong University, Shanghai (CN)

(21) Appl. No.: 18/575,298

(22) PCT Filed: Jun. 30, 2022
(86) PCT No.: PCT/CN2022/102857

§ 371 Date: Jul. 3, 2024

(30) Foreign Application Priority Data
Jul. 2, 2021 (CN) 202110751014.7
Jul. 5, 2021 (CN) 202110756337.5
Jul. 9, 2021 (CN) 202110780476.1
Jul. 27, 2021 (CN) 202110852545.5

(51) Int. Cl. G01N 33/68; H01J 49/04; H01J 49/16

(52) U.S. Cl. CPC G01N 33/6851; H01J 49/0463; H01J 49/164; G01N 2458/00

(57) ABSTRACT

A synthesis and preparation method for a nano-enhanced chip and its metal nano-composite materials required for laser desorption ionization mass spectrometry detection of small molecule metabolites are provided. The specific preparation method comprises: mixing basic chemical materials based on the chemical composition, size, and surface properties of the chip material. Ultrasonic, water bath, and centrifugation treatments are performed based on the properties of the material. A method for detecting small molecule metabolites based on the nano-enhanced chip is provided, wherein the chip is added as a matrix to proportionally diluted fluid samples, dried, and subjected to laser desorption ionization mass spectrometry analysis to obtain detection results.

### TECHNICAL FIELD

The disclosure relates to the field of composite nanomaterial chip synthesis, and in particular to a nano-enhanced chip and the use thereof in laser desorption ionization mass spectrometry detection of small molecule metabolite.

### DESCRIPTION OF RELATED ART

Biomarker (for example, protein, nucleic acid, and metabolite) detection plays an increasingly important role in in vitro diagnosis due to the non-invasive characteristics. Unlike genes whose functions are epigenetically regulated and post-translationally modified proteins, metabolites are direct markers of biochemical activity and are easily associated with phenotypes. Macromolecular substances such as nucleic acids and proteins can be used for disease diagnosis by amplifying trace amounts of substances to detectable levels with the support of technologies such as polymerase chain reaction and fluorescence enhancement. Compared with the macromolecular substances, signal amplification for small molecule metabolites still faces huge challenges, and high-sensitivity detection methods for small molecule metabolites need to be developed.

Currently, the detection of small molecule metabolites mainly relies on two major technologies, mass spectrometry and nuclear magnetic resonance (NMR) spectroscopy. Due to inherent limitations in the sensitivity and molecular identification of NMR spectroscopy, mass spectrometry is considered the primary detection technology for small molecule metabolites. The gas chromatography/liquid chromatography-mass spectrometry is the most important mass spectrometry method for detection of metabolites. However, this method requires complex preprocessing of samples such as desalting, protein removal, derivatization, and concentration to achieve detection of metabolites. It also requires a large sample size, and is time-consuming. Matrix-assisted laser desorption ionization mass spectrometry (MALDI MS) introduces matrix materials for photon absorption to promote the conversion of the substance to be measured from the solid phase to the gas phase, and a high-sensitivity and high-throughput detection of a trace amount of the substance to be measured can be directly achieved.

The performance of MALDI MS detection is subjected to the matrix material. Conventional organic matrices tend to produce strong background signals at an end of small molecular weight (m/z<400), the noises bring significant interference to the detection of small molecules, and thus the effect of detection is affected. In addition, there are various biological macromolecules in complex biological samples, and different pH and high salinity hinder the detection of small molecules. Therefore, it is hard for the conventional organic matrices to meet the needs of the detection of small molecules. Although conventional inorganic nanomaterials (such as carbon-based, silicon-based, and noble metal materials) may be used for the detection of small molecule metabolites, limitations during performing the detection of complex biological samples still exist.

### SUMMARY

The purpose of the disclosure is to develop a nano-enhanced chip and the use thereof in a laser desorption ionization mass spectrometry detection of a small molecule metabolite.

Specifically, the solutions involved in the disclosure include the following.
(I) A preparation method of a metal organic framework material and a use thereof in a detection of a small molecule is provided.
(II) A preparation method of a sub-micron reactor and a detection method of a serum metabolite based thereon is provided.
(III) A polygonal star-shaped Au@ZnO nanocomposite material, a preparation method thereof, and the use thereof are provided.
(IV) A preparation method of a porous alloy nanomaterial and a use thereof in a detection of a plasma metabolite.

That is to say, the nano-enhanced chip according to the disclosure may be the following metal organic framework material, sub-micron reactor, polygonal star-shaped Au@ZnO nanocomposite material, and porous alloy nanomaterial.

The purpose of the disclosure can be achieved through technical solutions as follows.
(I) A preparation method of a metal organic framework material and a use thereof in a detection of a small molecule is provided.

First, a preparation method of a metal organic framework material is provided. The method includes steps as follows.
Step 1: Dimethylformamide, ethanol, deionized water, triethylamine, and terephthalic acid are mixed and added into a closed container, and metal chloride is added to form a mixture.
Step 2: A water bath ultrasonic treatment is performed on the mixture to obtain a reactant.
Step 3: The reactant is centrifuged and washed to remove residual dimethylformamide to obtain a product.
Step 4: The product in step 3 is dispersed in deionized water, and an ultra-thin metal organic framework material is separated by using low-speed centrifugation.

Further, the metal chloride in step 1 is one of ferrous chloride, cobalt chloride, and nickel chloride, and an ultra-thin iron metal organic framework material, an ultra-thin cobalt metal organic framework material, and an ultra-thin nickel metal organic framework material are obtained respectively corresponding to step 4.

Further, the reaction condition for the water bath ultrasonic treatment in step 2 is at 0° C. for 4 hours.

Further, the washing method in step 3 is to wash five times each with deionized water and isopropyl alcohol.

Further, the ultra-thin iron metal organic framework material is further separated to obtain a multi-layer iron metal organic framework material.

Further, the ultra-thin nickel metal organic framework material is a single-layer nickel metal organic framework material.

Further, the disclosure also provides a use of the metal organic framework material in a detection of a small molecule.

Further, steps as follows are included.
Step 1: The detection mode of the matrix-assisted laser desorption ionization mass spectrometry is set to a positive ion reflection mode.
Step 2: The serum sample is diluted proportionally.
Step 3: Sample preparation is performed on the mass spectrometry target plate, the ultra-thin metal organic framework material is used as a matrix, and drying is performed at room temperature.
Step 4: The detection of the small molecule is performed on the serum sample.
Step 5: The original mass spectrum is analyzed and the detection result is obtained.

Further, the range of molecular mass of the small molecule in step 4 is less than 1000 Da.

Further, the small molecules in step 4 include, for example, amino acids and nucleosides.
(II) A preparation method of a sub-micron reactor and a detection method of a serum metabolite based thereon is provided.

The disclosure provides a preparation method of a sub-micron reactor, which includes steps as follows.
Step 2.1: 3-aminophenol APF is dissolved in deionized water, and formaldehyde solution and ammonia solution are added.
Step 2.2: The mixture in the step 2.1 is reacted at 30° C. for 30 minutes.
Step 2.3: The reactant in the step 2.2 is centrifuged and washed to obtain an APF submicron material.

Further, the reactant in step 2.2 is directly centrifuged and washed with deionized water to obtain a spherical APF submicron material APF-sphere.

Further, Acetone solution is added to the reactant in the step 2.2 to react at 30° C. for 180 minutes. The reactant is directly centrifuged and washed with deionized water to obtain a bowl-shaped APF submicron material APF-bowl.

Further, the obtained APF-sphere is dispersed in deionized water, and a chloroauric acid solution is added to react to obtain APF-sphere&Au.

Further, the added amount of the chloroauric acid solution is 2 ml.

Further, the obtained APF-bowl is dispersed in deionized water, and three reaction solutions are prepared and added into 1.5, 2, and 2.5 ml of chloroauric acid solution respectively to obtain APF-bowl&Au-1, APF-bowl&Au-2, and APF-bowl&Au-3.

Further, the reaction condition is at 70° C. for 10 minutes.

The disclosure also provides a detection method of a serum metabolite based on the sub-micron reactor, including steps as follows.
(1) Preparation of instruments and reagents: the detection mode of the matrix-assisted laser desorption ionization mass spectrometry is set to a positive ion reflection mode.
(2) The obtained APF-sphere&Au, APF-bowl&Au-1, APF-bowl&Au-2, and APF-bowl&Au-3 are dispersed in deionized water and used as matrices.
(3) The serum sample is diluted proportionally.
(4) Sample preparation is performed on the mass spectrometry target plate, the matrices in (2) are used, and drying is performed at room temperature.
(5) Detection of small molecule is performed on the serum sample.
(6) The original mass spectrum is analyzed and the detection result is obtained.

Further, the range of molecular mass detected is less than 1000 Da.

Further, substances detected include amino acids and sugar alcohols.
(III) A polygonal star-shaped Au@ZnO nanocomposite material, a preparation method thereof, and the use thereof are provided.

The disclosure provides a polygonal star-shaped Au@ZnO nanocomposite material to be used as a matrix material in a matrix-assisted laser desorption ionization mass spectrometry detection of a small molecule metabolite.

Preferably, the small molecule in the small molecule metabolite is selected from at least one type of proline, lysine, arginine, saccharose, and glucose.

The disclosure provides use of a polygonal star-shaped Au@ZnO nanocomposite material as a matrix material in a matrix-assisted laser desorption ionization mass spectrometry detection of a serum metabolite.

The disclosure provides a preparation method according to the polygonal star-shaped Au@ZnO nanocomposite material, which includes steps as follows.
(1) Preparation of a nanogold solution: a hydrated tetrachloroauric acid solution is stirred and heated to 120±1° C., sodium citrate dihydrate is added, stirred at a constant temperature for 30±0.1 min, and cooled to a room temperature for later use.
(2) Preparation of zinc oxide: a zinc acetate solution is added to a sodium hydroxide solution, heated to 60±1° C. and maintained for 1±0.1 h, centrifuged to collect precipitate, washed, and dried at 50±1° C. for later use.
(3) Preparation of the polygonal star-shaped Au@ZnO nanocomposite material: the zinc acetate solution is mixed with the nanogold solution to obtain a mixture, a sodium hydroxide solution is added to the mixture, heated to 60±1° C. and maintained for 1±0.1 h, centrifuged to collect precipitate, washed, and dried at 50±1° C. for later use.

Preferably, in step (1), the stirring speed is 800±10 rpm.

Preferably, in step (2) and step (3), the centrifuge speed is 1000±10 rpm.

The disclosure provides a polygonal star-shaped Au@ZnO nanocomposite material. which is obtained by the preparation method.
(IV) A preparation method of a porous alloy nanomaterial and the use of the porous alloy nanomaterial in a detection of a plasma metabolite are provided.

The technical problem to be solved by this disclosure is how to use a MALDI MS matrix with a good LDI efficiency in a detection of a metabolite of a complex biological sample (plasma). Based on above, the disclosure provides a preparation method of a porous alloy nanomaterial, including steps as follows.
Step 1: Na2PdCl4, H2PtCl6·6H20, hydrochloric acid, and F127 are fully mixed and dissolved with an ultrasonic treatment.
Step 2: After being completely dissolved, an ascorbic acid solution is added and immediately placed in a water bath ultrasonic treatment.
Step 3: Then, a HAuCl4·4H2O solution is added to react.
Step 4: Finally, a porous alloy nanomaterial PdPtAu is obtained by centrifuging and washing with absolute ethanol and water respectively, and then drying.

Further, the added amounts of Na2PdCl4, H2PtCl6·6H2O, hydrochloric acid, and F127 in step 1 are respectively 0.6 mL of 20 Mm Na2PdCl4, 3 mL of 20 Mm H2PtCl6·6H20, 60 μL of 6.0M hydrochloric acid, and 60 mg of F127.

Further, the added amount of the ascorbic acid solution in step 2 is 3 mL of 0.1M ascorbic acid solution.

Further, the water bath ultrasonic treatment in step 2 is at 45° C. for 3 hours.

Further, the concentration of the HAuCl4·4H20 solution in step 3 is 10 Mm to 40 Mm, and the added amount is 1.2 mL.

Further, the number of centrifugal washings in step 3 is 3 times, the rotation speed is 10000 rpm, and the drying temperature is 50° C.

In the use of the porous alloy nanomaterial in the detection of the plasma metabolite, the porous alloy nanomaterial is used as the matrix in the matrix-assisted laser desorption ionization mass spectrometry.

Further, steps as follows are included.
Step 1: The porous alloy nanomaterial PdPtAu is dispersed in deionized water.
Step 2: The plasma is mixed with an equal volume of methanol/acetonitrile mixture, wherein the volume ratio of methanol/acetonitrile is 1:1, is shaken on a shaker for 10 minutes and is centrifuged for 10 minutes, and the supernatant is taken for mass spectrometry detection.
Step 3: The matrix-assisted laser desorption ionization mass spectrometry sets to the positive ion reflection mode, and sample preparation is performed on the mass spectrometry target plate of the matrix-assisted laser desorption ionization mass spectrometry. The sample is 1 μL of plasma extract, and the detection is performed on the small molecule of the plasma sample.

Further, the molecular weight range of the small molecule is less than 1000 Da.

Further, the small molecule is carbohydrate or amino acid.

Compared with the existing technology, the disclosure has advantages and beneficial effects as follows.
(I) In the preparation method of the metal organic framework material and the use of the metal organic framework material in the detection of the small molecule according to the disclosure, metal ions (including iron, cobalt, nickel) and thickness (including ultra-thin and multi-layer) of the metal organic framework material can be precisely adjusted, the defect of the conventional matrix is overcome, and the detection of serum is carried out in a manner of low sample volume (0.1 μl), rapid detection (less than 1 minute), high-throughput (approximately 120,000 data points, more than 300 metabolic features), and high sensitivity. The metal organic framework material uses transition metals. Compared with precious metals, the preparation cost is lower and has superior cost-effectiveness. Also, the preparation may be realized by a one-step method and the synthesis step is simple. By optimizing a series of metal organic framework materials, the optimized ultra-thin iron metal organic framework material provides a large number of active sites for molecular detection. When being used as a matrix material in MALDI-TOF-MS detection, the existing defects of conventional organic matrices such as interference in the low molecular weight range (m/z<1000) and hot spot effect can be solved, and efficient analysis of serum sample is achieved. The serum sample merely needs to undergo simple, efficient, and rapid detection to analyze the small molecule metabolite in the serum.
(II) In the preparation method of the sub-micron reactor and the detection method of the serum metabolite based thereon according to the disclosure, the preparation steps of a series of sub-micron reactor chip materials are simple, the synthesis process is safe, and the yield is large, which has superior cost-effectiveness. The optimized sub-micron reactor chip material is used as the matrix material for laser desorption ionization time-of-flight mass spectrometry. Compared with conventional organic matrix materials, sensitive detection of small molecule metabolite substance in serum can be achieved in the low molecular weight range (m/z<400). The optimized sub-micron reactor chip material can achieve high-throughput (approximately 120,000 data points, more than 300 metabolic features) and rapid (less than 1 minute) detection of serum using merely 0.1 μL of serum sample. Based on the above significant advantages, the sub-micron reactor chip materials are expected to realize large-scale clinical detection of serum and be used in treatment detection to screen out corresponding metabolic biomarkers for therapeutic efficacy.
(III) In the polygonal star-shaped Au@ZnO nanocomposite material, the preparation method thereof, and the use thereof according to the disclosure, compared with organic matrices, using Au@ZnO nanocomposite material as the matrix for laser desorption ionization mass spectrometry can solve the problems of conventional organic matrices such as hot spot effect and interference in the low molecular weight range. Compared with the inorganic matrix, the Au@ZnO nanocomposite material of the disclosure can combine the advantages of both precious metals and semiconductor materials, improve the hot carrier generation effect through the Schottky effect, and has a high degree of controllability. The synergy effect improves detection efficiency and reduces costs. Compared with conventional core-shell nanomaterials or spherical nanomaterials, the special polygonal star-shaped composite nanoparticles of the disclosure enhance the electromagnetic field at the tips and depressions, which helps enhance surface plasmon resonance and improve the laser desorption ionization effect and at the same time helps increase the specific surface area and enhance the adsorption of small molecule metabolite. Therefore, the polygonal star-shaped Au@ZnO composite nanomaterial is used for the detection of small molecule metabolite and serum metabolite in LDI MS.
(IV) In the preparation method of the porous alloy nanomaterial and the use thereof in the detection of the plasma metabolite according to the disclosure, the porous PdPtAu alloy can be synthesized through a one-step method with simple synthesis steps and low preparation cost. As a matrix for laser desorption ionization mass spectrometry, the nanomaterial can solve the problems of conventional organic matrices such as hot spot effect and interference in low molecular weight range. In the disclosure, the plasma sample merely needs to undergo simple pretreatment, and each sample merely requires 1 μL of plasma extraction to perform a rapid and sensitive detection of small molecule metabolite in plasma. The method has high accuracy, low cost, and high throughput of detection, which meets the needs of clinical detection of plasma.

### BRIEF DESCRIPTION OF THE DRAWINGS

FIG. 1 is a scanning electron microscope characterization image of an ultra-thin iron metal organic framework prepared according to a preferred embodiment of a preparation method of the metal organic framework material and the use in the detection of the small molecule.

FIG. 2 is a mass spectrum of the metal organic framework in matrix-assisted laser desorption ionization mass spectrometry detection of proline standard molecule prepared according to an embodiment of the preparation method of the metal organic framework material and the use thereof in the detection of the small molecule.

FIG. 3 is a mass spectrum of the metal organic framework in matrix-assisted laser desorption ionization mass spectrometry detection of creatinine standard molecule prepared according to a preferred embodiment of the preparation method of the metal organic framework material and the use thereof in the detection of the small molecule.

FIG. 4 is a mass spectrum of the metal organic framework in matrix-assisted laser desorption ionization mass spectrometry detection of serum low molecular weight segment prepared according to a preferred embodiment of the preparation method of the metal organic framework material and the use thereof in the detection of the small molecule.

FIG. 5 is a mass spectrum of the metal organic framework in matrix-assisted laser desorption ionization mass spectrometry detection of small molecules of different serum samples prepared according to a preferred embodiment of the preparation method of the metal organic framework material and the use thereof in the detection of the small molecule, and the serum samples are detected and identified as the disease group versus the control group in MATLAB.

FIG. 6 is a scanning electron microscope image of APF-sphere according to a preferred embodiment of a preparation method of a sub-micron reactor and a detection method of serum metabolite based thereon.

FIG. 7 is a transmission electron microscope characterization image of APF-bowl according to a preferred embodiment of the preparation method of the sub-micron reactor and the detection method of serum metabolite based thereon.

FIG. 8 is a scanning electron microscope characterization image of APF-sphere&Au according to a preferred embodiment of the preparation method of the sub-micron reactor and the detection method of serum metabolite based thereon.

FIG. 9 is a scanning electron microscope characterization image of APF-bowl&Au according to a preferred embodiment of the preparation method of the sub-micron reactor and the detection method of serum metabolite based thereon.

FIG. 10 shows statistical histogram results of five independent experiments for MALDI-TOF-MS detection of leucine standard molecules by using a series of sub-micron reactor chip materials (including APF-sphere&Au, APF-bowl&Au-1, APF-bowl&Au-2, and APF-bowl&Au-3) according to Example 1 of the preparation method of the sub-micron reactor and the detection method of serum metabolite based thereon.

FIG. 11 shows statistical histogram results of five independent experiments for MALDI-TOF-MS detection of mannitol standard molecules by using a series of sub-micron reactor chip materials (including APF-sphere&Au, APF-bowl&Au-1, APF-bowl&Au-2, and APF-bowl&Au-3) according to Example 2 of the preparation method of the sub-micron reactor and the detection method of serum metabolite based thereon.

FIG. 12 is a mass spectrum of matrix-assisted laser desorption ionization mass spectrometry detection of serum low molecular weight segment according to Example 3 of the preparation method of the sub-micron reactor and the detection method of serum metabolite based thereon.

FIG. 13 is a mass spectrum of matrix-assisted laser desorption ionization mass spectrometry detection of small molecules of different serum samples monitored as before chemotherapy versus after chemotherapy in MATLAB according to Example 4 of the preparation method of the sub-micron reactor and the detection method of serum metabolite based thereon.

FIG. 14 is a characterization image of ZnO in the related art and a polygonal star-shaped Au@ZnO nanocomposite material according to the disclosure (a of FIG. 14 is a scanning electron microscope image of ZnO nanoparticles in the related art, b of FIG. 14 is a scanning electron microscope image of the polygonal star-shaped Au@ZnO nanocomposite material of the disclosure, c of FIG. 14 is an analysis chart of a line scan energy dispersive x-ray (EDX) of the polygonal star-shaped Au@ZnO nanocomposite material of the disclosure, d of FIG. 14 is an element distribution diagram of the polygonal star-shaped Au@ZnO nanocomposite material of the disclosure, e of FIG. 14 is an EDX result diagram of ZnO and the polygonal star-shaped Au@ZnO nanocomposite material of the disclosure, f of FIG. 14 is the UV-visible absorption spectra of Au, ZnO, and the polygonal star-shaped Au@ZnO nanocomposite material of the disclosure, and g of FIG. 14 is an electron diffraction pattern of a selected area of the polygonal star-shaped Au@ZnO nanocomposite material of the disclosure).

FIG. 15 shows mass spectra of four standard small molecules in the MALDI MS detection of the polygonal star-shaped Au@ZnO nanocomposite material according to an embodiment of the disclosure (a of FIG. 15 shows glucose, b of FIG. 15 shows saccharose, c of FIG. 15 shows proline, and d of FIG. 15 shows lysine).

FIG. 16 shows mass spectra of four standard small molecules and serum metabolites in the MALDI MS detection of the polygonal star-shaped Au@ZnO nanocomposite material according to an embodiment of the disclosure (a of FIG. 16 shows a mixture of four metabolites containing sodium chloride, b of FIG. 16 shows a mixture of four metabolites containing bovine serum albumin, c of FIG. 16 shows a mixture of four metabolites containing CHCA, d of FIG. 16 shows a mixture of four metabolites containing CHCA, and FIG. 16e is a mass spectrum detection chart of standard serum).

FIG. 17 is a scanning electron microscope characterization image of a porous PdPtAu nanomaterial according to a preferred embodiment of a preparation method of a porous alloy nanomaterial and use thereof in the detection of the plasma metabolite.

FIG. 18 is a transmission electron microscope characterization image of the porous PdPtAu nanomaterial according to a preferred embodiment of the preparation method of the porous alloy nanomaterial and the use thereof in the detection of the plasma metabolite.

FIG. 19 is a mass spectrum of matrix-assisted laser desorption ionization mass spectrometry detection of standard small molecule using the porous PdPtAu nanomaterial as the matrix according to a preferred embodiment of the preparation method of the porous alloy nanomaterial and the use thereof in the detection of the plasma metabolite.

FIG. 20 is a mass spectrum of a matrix-assisted laser desorption ionization mass spectrometry detection of a small molecule containing a) NaCl and b) bovine serum albumin using the porous PdPtAu nanomaterial as the matrix according to a preferred embodiment of the preparation method of the porous alloy nanomaterial and the use thereof in the detection of the plasma metabolite.

FIG. 21 is a mass spectrum of matrix-assisted laser desorption ionization mass spectrometry detection of small molecule in plasma sample using the porous PdPtAu nanomaterial as the matrix according to a preferred embodiment of the preparation method of the porous alloy nanomaterial and the use thereof in the detection of the plasma metabolite.

### DESCRIPTION OF THE EMBODIMENTS

The disclosure will be described in detail below with reference to the accompanying drawings and specific embodiments.
(I) Examples of the preparation method of the metal organic framework material and the use thereof in the detection of the small molecule are provided.

### Preparation Method Example 1

Step 1: Preparation of instruments and reagents: the detection mode of the matrix-assisted laser desorption ionization mass spectrometry is set to a positive ion reflection mode.
Step 2: A series of metal organic framework matrices are prepared, including steps as follows.
Step 2.1: Dimethylformamide, ethanol, deionized water, triethylamine, and terephthalic acid are mixed and added into a closed container, and gold ferrous chloride, cobalt chloride, or nickel chloride are added respectively.
Step 2.2: The mixture in step 2.1 is reacted with a water bath ultrasonic treatment at 0° C. for 4 hours.
Step 2.3: The reactant in step 2.2 is centrifuged and washed five times each with deionized water and isopropyl alcohol to remove residual dimethylformamide.
Step 2.4: The product in step 2.3 is dispersed in deionized water and an ultra-thin metal organic framework material (MOF-UL) is separated by using low-speed centrifugation. According to the differences in metal chlorides, ultra-thin iron metal organic framework material (Fe-MOF-UL), ultra-thin cobalt metal organic framework material (Co-MOF-UL), and single-layer nickel metal organic framework material (Ni-MOF-UL) are obtained respectively.
Step 2.5: A multi-layer iron metal organic framework material (Fe-MOF-Bulk) is separated from the sediment in step 2.4.
Step 2.6: The Fe-MOF-UL, Co-MOF-UL, or Ni-MOF-UL and Fe-MOF-Bulk obtained in step 2.4 and step 2.5 are dispersed in deionized water and used as a matrices.
Step 3: The serum sample is diluted proportionally.
Step 4: Sample preparation is performed on the mass spectrometry target plate, the optimized Fe-MOF-UL is used as the matrix, and drying is performed at room temperature.
Step 5: Detection of small molecule is performed on the serum sample.
Step 6: The original mass spectrum is analyzed and the detection result is obtained.

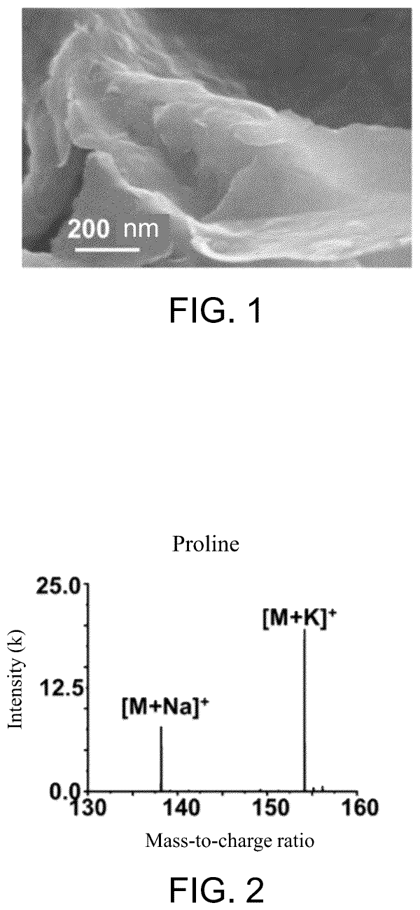

As shown in FIG. 1, a Hitachi S-4800 scanning electron microscope is used to obtain the transmission electron microscopy result. The prepared ultra-thin iron metal organic framework material has a layered structure, and the synthesized material has a small thickness and a uniform surface structure.

### Application Example 1

### Detection of Proline Standard

(1) Preparation of instruments and reagents: the detection mode of the matrix-assisted laser desorption ionization mass spectrometry is set to a positive ion reflection mode, and the prepared MOF material and a prepared proline standard solution are prepared.
(2) Sample preparation is performed on the mass spectrometry target plate, and drying is performed at room temperature.
(3) Detection is performed using the mass spectrometry, and the mass spectrum image is analyzed. The result is shown in FIG. 2.

### Application Example 2

### Detection of Creatinine Standard

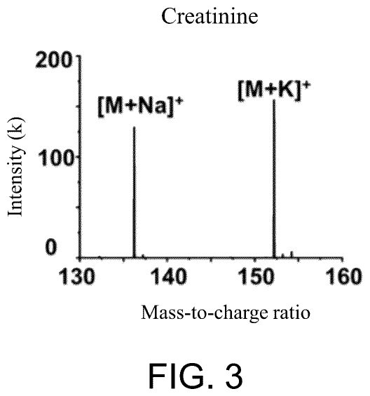

(1) Preparation of instruments and reagents: the detection mode of the matrix-assisted laser desorption ionization mass spectrometry is set to a positive ion reflection mode, and the prepared MOF material and a prepared creatinine standard solution are prepared.
(2) Sample preparation is performed on the mass spectrometry target plate, and drying is performed at room temperature.
(3) Detection is performed using the mass spectrometry, and the mass spectrum image is analyzed. The result is shown in FIG. 3.

### Application Example 3

### Detection of Small Molecule of Serum Sample

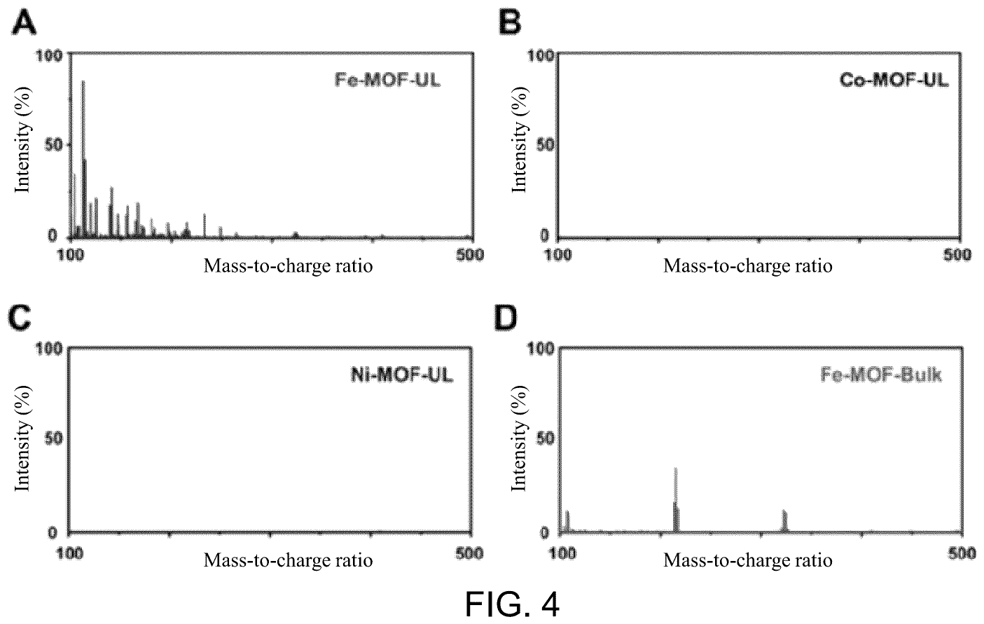

(1) Preparation of instruments and reagents: the detection mode of the matrix-assisted laser desorption ionization mass spectrometry is set to a positive ion reflection mode, and the series of metal organic framework materials prepared, including Fe-MOF-UL, Co-MOF-UL, Ni-MOF-UL, and Fe-MOF-Bulk are prepared.
(2) The serum sample is diluted according to a certain ratio.
(3) Sample preparation is performed on the mass spectrometry target plate, and drying is performed at room temperature.
(4) Detection is performed using the mass spectrometry, and the mass spectrum image is analyzed. The result is shown in FIG. 4.

### Application Example 4

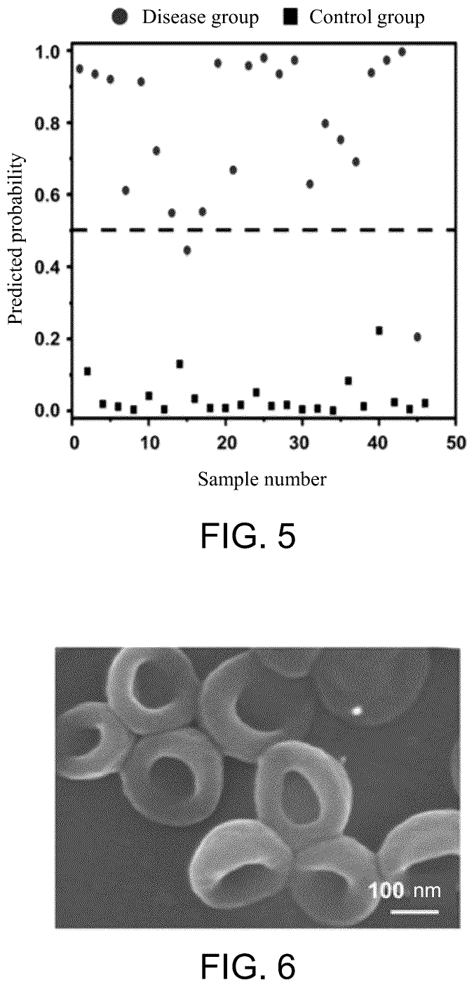

Detection and identification of serum samples from the disease group and the control group are performed, in which the disease group consists of 23 patient serum samples, and the control group consists of 23 healthy volunteer serum samples.
(1) Preparation of instruments and reagents: the detection mode of the matrix-assisted laser desorption ionization mass spectrometry is set to a positive ion reflection mode, and the prepared MOF material and MATLAB analysis software are prepared.
(2) The serum sample is diluted according to a certain ratio.
(3) Sample preparation is performed on the mass spectrometry target plate, and drying is performed at room temperature.
(4) Detection is performed using the mass spectrometry, and mass spectrometry data is collected.
(5) The mass spectrometry data is preprocessed and analysis is performed using MATLAB analysis software. The result is shown in FIG. 5.
(II) Examples of the preparation method of the sub-micron reactor and the detection method of the serum metabolite based thereon are provided.

The technical solution is as follows.
Step 1: Preparation of instruments and reagents: the detection mode of the matrix-assisted laser desorption ionization mass spectrometry is set to a positive ion reflection mode.
Step 2: The sub-micron reactor is prepared, including steps as follows.
Step 2.1: 0.1 g of 3-aminophenol (APF) is dissolved in 30 ml of deionized water, and 0.1 ml of formaldehyde solution and 0.1 ml of ammonia solution are added.
Step 2.2: The mixture in step 2.1 is reacted at 30° C. for 30 minutes.
Step 2.3: The reactant in step 2.2 is centrifuged and washed five times with deionized water to obtain a spherical APF submicron material (APF-sphere).
Step 2.4: 40 ml of acetone solution is added to the reactant in step 2.2 and reacted for at 30° C. for 180 minutes.
Step 2.5: The reactant in step 2.4 is centrifuged and washed five times with deionized water to obtain a bowl-shaped APF submicron material (APF-bowl).
Step 2.6: 10 mg of the APF-sphere obtained in step 2.3 is dispersed in 10 ml of deionized water, and 2 ml of chloroauric acid solution with a mass concentration of 1% is added.
Step 2.7: 10 mg of the APF-bowl obtained in step 2.5 is dispersed in 10 ml of deionized water, three portions of the reaction solution are prepared, and 1.5, 2, and 2.5 ml of chloroauric acid solution with a mass concentration of 1% are added respectively.
Step 2.8: The reactants obtained in step 2.6 and step 2.7 are reacted at 70° C. for 10 minutes, centrifuged and washed with water, and APF-sphere&Au, APF-bowl&Au-1, APF-bowl&Au-2, and APF-bowl&Au-3 are obtained sequentially.
Step 2.9: The APF-sphere&Au, APF-bowl&Au-1, APF-bowl&Au-2, and APF-bowl&Au-3 obtained in step 2.8 are dispersed in deionized water and used as matrices.
Step 3: The serum sample is diluted proportionally with a 10-fold dilution.
Step 4: Sample preparation is performed on the mass spectrometry target plate, the matrix in step 2.9, preferably APF-bowl&Au-2, is adopted, and drying is performed at room temperature.
Step 5: Detection of small molecule is performed on the serum sample.
Step 6: The original mass spectrum is analyzed and the detection result is obtained.

Further, the performance of mass spectrometry detection using the APF-bowl&Au-2 with the bowl-shaped structure obtained by the reaction of 2 ml of chloroauric acid is good. The molecular weight range of detection is less than 1000 Da. Substances detected include amino acids and sugar alcohols.

### Instruments Used for Characterization

A Hitachi S-4800 scanning electron microscope is used to obtain the SEM result. A JEOL JEM-2100F transmission electron microscope is used to obtain the TEM result.

The characterization results are as follows.

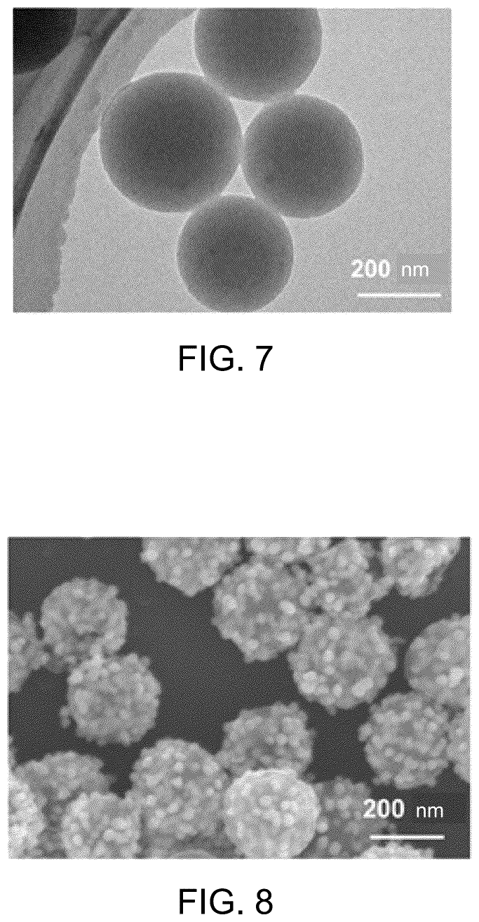

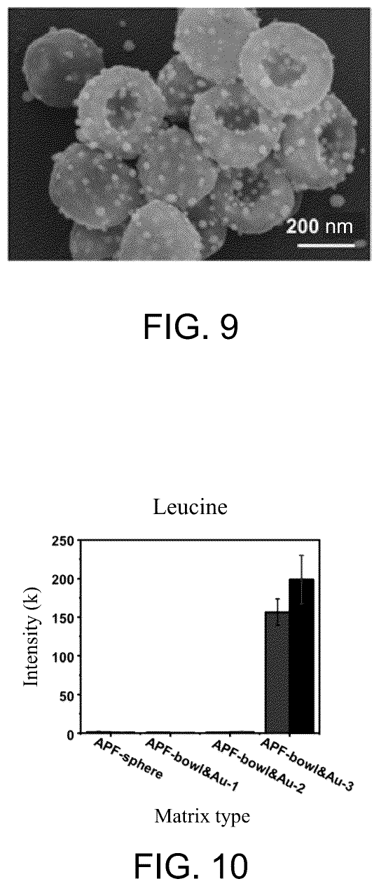

As shown in FIG. 6 to FIG. 9, the prepared APF-sphere and APF-bowl have smooth surfaces and uniform shapes, while the APF-sphere&Au and APF-bowl&Au modified with chloroauric acid have rough surfaces, in which notable gold particles may be seen.

### Example 1: Detection of Leucine Standard

Preparation of instruments and reagents: the detection mode of the matrix-assisted laser desorption ionization mass spectrometry is set to a positive ion reflection mode. The prepared sub-micron reactor matrix materials, including APF-sphere&Au, APF-bowl&Au-1, APF-bowl&Au-2, and APF-bowl&Au-3 are prepared. A leucine standard solution is prepared. Sample preparation is performed on the mass spectrometry target plate, and drying is performed at room temperature. Detection is performed using the mass spectrometry, and the mass spectrum image is analyzed. The result is shown in FIG. 10.

### Example 2: Detection of Mannitol Standard

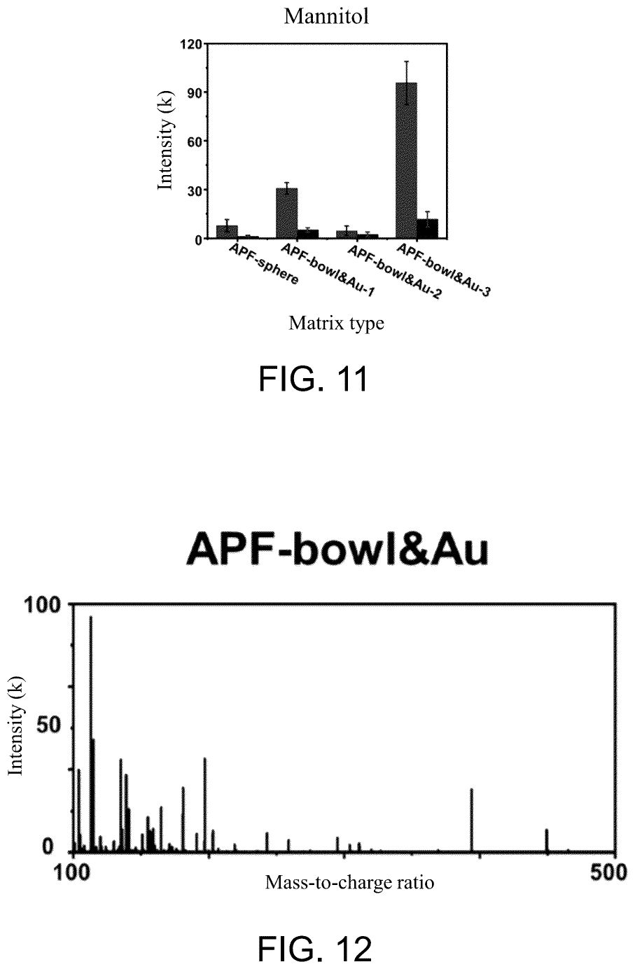

Preparation of instruments and reagents: the detection mode of the matrix-assisted laser desorption ionization mass spectrometry is set to a positive ion reflection mode. The prepared sub-micron reactor matrix materials, including APF-sphere&Au, APF-bowl&Au-1, APF-bowl&Au-2, and APF-bowl&Au-3 are prepared. A mannitol standard solution is prepared. Sample preparation is performed on the mass spectrometry target plate, and drying is performed at room temperature. Detection is performed using the mass spectrometry, and the mass spectrum image is analyzed. The result is shown in FIG. 11.

### Example 3: Detection of Small Molecule of Serum Sample

Preparation of instruments and reagents: the detection mode of the matrix-assisted laser desorption ionization mass spectrometry is set to a positive ion reflection mode. The prepared optimized sub-micron reactor APF-bowl&Au-2 is used as the matrix material. The serum sample is diluted according to a certain proportion. Sample preparation is performed on the mass spectrometry target plate, and drying is performed at room temperature. Detection is performed using the mass spectrometry, and the mass spectrum image is analyzed. The result is shown in FIG. 12.

### Example 4: Detection and Monitoring of Serum Samples Before and After Chemotherapy

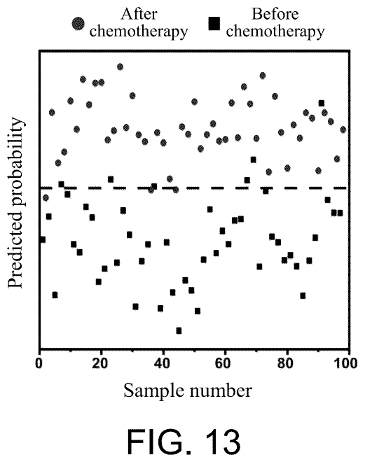

Preparation of instruments and reagents: the detection mode of the matrix-assisted laser desorption ionization mass spectrometry is set to a positive ion reflection mode. The prepared optimized sub-micron reactor APF-bowl&Au-2 is used as matrix material. MATLAB and Metaboanalyst analysis software are prepared. The serum sample is diluted according to a certain ratio. Sample preparation is performed on the mass spectrometry target plate, and drying is performed at room temperature. Detection is performed using the mass spectrometry, and mass spectrometry data is collected. The mass spectrometry data is preprocessed and analysis is performed using MATLAB analysis software. The result is shown in FIG. 13.

A new type of the sub-micron reactor chip according to the disclosure is used as a matrix to assist matrix-assisted laser desorption ionization time-of-flight mass spectrometry (MALDI-TOF-MS) to improve the detection performance thereof, especially for complex serum samples. By adjusting the morphological structure (spherical, bowl-shaped) and composition (gold loading content) of sub-micron reactor chip materials, the defect of conventional matrices can be overcome, and a rapid, high-throughput, and high-sensitivity detection of small molecules metabolite in serum is achieved.

Sub-micron reactor chip materials are easy to prepare, the synthesis process is safe, and the yield is high. By optimizing the sub-micron reactor chip materials, the optimized APF-bowl&Au loaded with gold nanoparticles is used as the matrix material in the MALDI-TOF-MS detection, which can solve the existing defects of conventional organic matrices such as interference in the low molecular weight segment and hot spot effect, and efficient analysis of serum sample is achieved. In the disclosure, the sub-micron reactor chip material can assist mass spectrometry in realizing detection of small molecule metabolite of serum sample, and the method for detection has high sensitivity and high throughput, which has the potential to be used in clinical applications.
(III) Examples of the polygonal star-shaped Au@ZnO nanocomposite material, the preparation method thereof, and the use thereof are provided.

This embodiment provides the polygonal star-shaped Au@ZnO nanocomposite material. the preparation method thereof, and the use thereof.

The polygonal star-shaped Au@ZnO nanocomposite material of the disclosure can be used as a matrix material in MALDI MS detection. Furthermore, the polygonal star-shaped Au@ZnO nanocomposite material of the disclosure is used as a matrix material in MALDI MS detection of the small molecule metabolite, in which the small molecule in the small molecule metabolite is selected from at least one type of proline, lysine, arginine, saccharose, and glucose.

Furthermore, the polygonal star-shaped Au@ZnO nanocomposite material of the disclosure is used as a matrix material in MALDI MS detection of the metabolite in serum.

The preparation method of the polygonal star-shaped Au@ZnO nanocomposite material according to the disclosure includes steps as follows.
(1) Use the citric acid reduction method to synthesize a nanogold solution: a hydrated tetrachloroauric acid solution is stirred and heated to 120±1° C., sodium citrate dihydrate is added, stirred at a constant temperature for 30±0.1 min, and cooled to a room temperature for later use.
(2) Preparation of zinc oxide: a zinc acetate solution is added to a sodium hydroxide solution, stirred and heated to 60±1° C. and maintained for 1±0.1 h, discarded the supernatant, washed three times each with ethanol and water, and dried at 50±1° C. for later use.
(3) Preparation of the polygonal star-shaped Au@ZnO nanocomposite material: the zinc acetate solution is mixed with the nanogold solution to obtain a mixture, a sodium hydroxide solution is added to the mixture, continued to stir and heat to 60±1° C. and maintained for 1±0.1 h. discarded the supernatant, wash three times each with ethanol and water, and dried at 50±1° C. for later use.

In step (1), the stirring speed is 800±10 rpm. In step (2) and step (3), the centrifuge speed is 1000±10 rpm.

The polygonal star-shaped Au@ZnO nanocomposite material of the disclosure is obtained by the preparation method.

In summary, the polygonal star-shaped Au@ZnO nanocomposite material of the disclosure is used as a matrix in matrix-assisted laser desorption ionization mass spectrometry detection of the small molecule metabolite and the serum metabolite. First of all, since semiconductor zinc oxide and metallic gold are combined, the photoelectric effect and the hot carrier generation efficiency are improved through the Schottky effect, which helps in the ionization of the object to be measured. Secondly, due to the special polygonal star-shaped form, the electromagnetic field is enhanced at the tips and depressions, which helps enhance surface plasmon resonance while increases the specific surface area, which helps adsorb small molecules of metabolites to be detected. Finally, the nanocomposite material has strong absorption at the wavelength of 355 nm, which matches the light source wavelength of the matrix-assisted laser desorption ionization mass spectrometry. Therefore, through simple pretreatment, the sample processing step is simplified, the LDI efficiency is improved, and the complex sample pretreatment process required by conventional GC/LC MS is overcome. Also, the defects of the conventional LDI MS matrix is overcome, and a rapid, sensitive, and high-throughput detection of small molecule metabolite and serum metabolite is achieved.

The preparation method of the polygonal star-shaped Au@ZnO nanocomposite material of the disclosure is simple and the morphology is uniform. As the matrix of LDI MS, the nanoparticles can solve the problems existing in the conventional matrix, such as background interference in the low molecular weight segment. In the disclosure, the serum sample merely requires 1.5 μL of serum extraction to perform a detection of small molecule metabolite in the serum efficiently and rapidly. The detection method has high sensitivity, low cost, and high detection throughput, which meets the needs of clinical detection of serum and has the potential to be used in clinical applications.

### Instruments Used for Characterization

The extinction spectra and surface charge values of nanomaterials are obtained using AuCy UV1900 spectrophotometer and Malvern Zetasizer NanZS90.

Hitachi SU8100 is used to obtain the scanning electron microscopy image, and JEOL JEM-2100F is used to obtain the transmission electron microscopy image, line scan energy dispersive X-ray spectra, and selected electron diffraction patterns.

The characterization results are as follows.

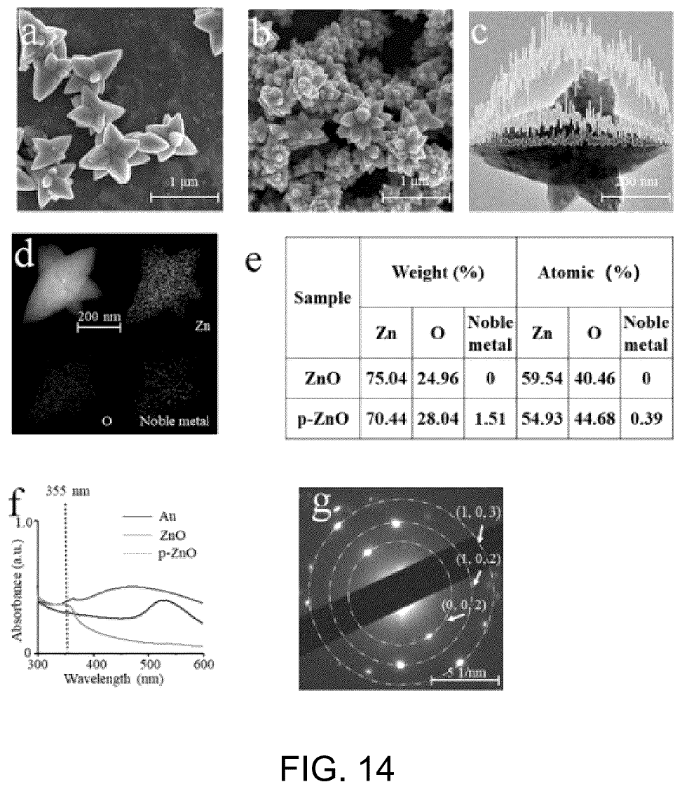

It can be seen from a of FIG. 14 and b of FIG. 14 that both nano-ZnO and nano-Au@ZnO have polygonal star-shaped structures. Au@ZnO (b of FIG. 14) significantly has more angles and a more uniform morphology. From the line scan analysis (c of FIG. 14, Zn, O and Au from top to bottom) and the element distribution analysis (d of FIG. 14), the obtained Au@ZnO contains three elements, Zn, O, and Au, proving the effect of nanogold on the morphology of zinc oxide. Due to the small particle size of the metal seeds (30 nm), a polygonal star-shaped structure like petals is formed. Energy dispersive X-ray spectroscopy shown in e of FIG. 14 further documents an increase in metal content from 0% to 1.51% (by weight), indicating that the result is consistent with other characterizations. UV-visible spectrum analysis shows that Au@ZnO has the highest absorption at 355 nm, which is consistent with the wavelength of the Nd:YAG laser of mass spectrometry and is beneficial to mass spectrometry detection (f of FIG. 14). Further, the crystal structure of the p-ZnO composite is characterized, correlating with the [1,0,3], [1,0,2], and [0,0,2] crystal planes of leucoferite (g of FIG. 14). In summary, the plasmonic ZnO composite material has a unique structure and composition and has the potential for LDI MS detection.

The preparation method of the polygonal star-shaped Au@ZnO nanocomposite material according to an embodiment includes steps as follows.
(1) Preparation of a nanogold solution: 10.45 mg hydrated tetrachloroauric acid is dissolved in deionized water in a 100 mL flask, heated to 120° C. with vigorous stirring (800 rpm), then added 2.0 mL with 1 wt % sodium citrate dihydrate, maintained stirring at 120° C. for 30 minutes, and then cooled to room temperature for later use.
(2) Preparation of ZnO: a zinc acetate solution (39 mL, 0.01 mol/L) is added to a sodium hydroxide solution (65 mL, 0.03 mol/L), heated to 60° C. and maintained 1 hour, then centrifuged at 10,000 rpm for 10 minutes, collected the precipitate, discarded the supernatant, and washed three times each with ethanol and water. The obtained precipitate is dried in a 50° C. oven for later use.
(3) Preparation of the polygonal star-shaped Au@ZnO nanocomposite material: the zinc acetate solution (39 mL, 0.01 mol/L) and 39 mL nanogold solution are mixed and stirred for 2 minutes (600 rpm) to obtain a mixture, added a sodium hydroxide solution (65 mL, 0.03 mol/L) to the mixture, continued to stir and heat to 60° C. and maintained for 1 hour, then centrifuged at 10,000 rpm for 10 minutes, collect the precipitate, discard the supernatant, and washed three times each with ethanol and water. The obtained precipitate is dried in a 50° C. oven for later use.

The polygonal star-shaped Au@ZnO nanocomposite material obtained in the Example is used to perform the following experiments.

### LDI MS Detection:

Detection of the small molecule metabolite: the standard small molecule (proline, lysine, saccharose, and glucose) are prepared with ultrapure water as a 1 mg/mL solution.

The salt-tolerant detection sample is made by adding sodium chloride to a mixture of four standard small molecules (proline, lysine, arginine, and glucose). The final concentrations of sodium chloride and small molecule are 0.2 mg/mL and 1 mg/mL respectively. For protein-resistant detection, the detection sample is a mixture of albumin and standard small molecules (proline, lysine, arginine, and glucose). The final concentrations of albumin and small molecules are both 1 mg/mL. The effects of detection at high salt concentrations and proteins using different matrices are explored.

In a typical LDI MS experiment, the Au@ZnO nanocomposite material is dispersed in water at a concentration of 1 mg/mL as the matrix. For the case where Au nanoparticles and ZnO nanoparticles are used as the matrix, the nanoparticles are also dispersed in water at a concentration of 1 mg/mL. Drop 1.5 μL of analyte solution (standard small molecule solution or standard serum) onto the polished target plate, dried at room temperature, and covered with 1.5 μL of matrix suspension. After drying. LDI mass spectrometry analysis is performed. An AutoFlex TOF/TOF mass spectrometry (Bruker, Germany) equipped with a Nd: YAG laser (2 kHz, 355 nm) is used for the mass spectrometry detection. Acquisition is performed in a positive ion reflection mode using delayed extraction with a repetition frequency of 1000 Hz and an accelerating voltage of 20 kV. The delay time of this experiment is optimized to 250 ns. In all LDI MS experiments, the number of laser shots per analysis is 2000.

### Mass Spectrometry Detection of Small Molecule Substances and Salt and Protein Tolerance Testing

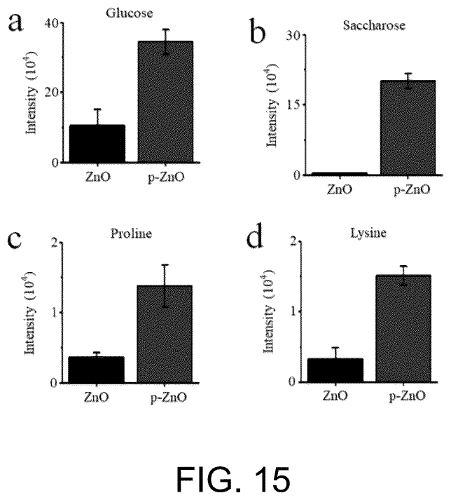

(1) Preparation of instruments and reagents: matrix-assisted laser desorption ionization mass spectrometry is set to a positive ion reflection mode for detection. The Au@ZnO nanocomposite material is prepared into a suspension, and a single standard small molecule (glucose, saccharose, proline, and lysine) solution, a mixed standard (proline, lysine, arginine, and glucose) solution, and a mixed solution of high concentration salt and protein are prepared.
(2) Sample preparation is performed on the mass spectrometry target plate, and drying is performed at room temperature.
(3) Detection is performed using the mass spectrometry, and the mass spectrum image is analyzed. The result is shown in FIG. 15. The detection effect of the polygonal star-shaped Au@ZnO nanocomposite material according to this embodiment, that is, the four typical small molecule metabolites (glucose, saccharose, proline, and lysine)of p-ZnO, is better than the effect of the pure zinc oxide (ZnO) nanoparticles, especially for saccharose, in which the mass spectrum signal difference is more than 17 times. Therefore, it may be said that the polygonal star-shaped Au@ZnO nanocomposite material (p-ZnO) is more suitable for LDI mass spectrometry detection than zinc oxide.

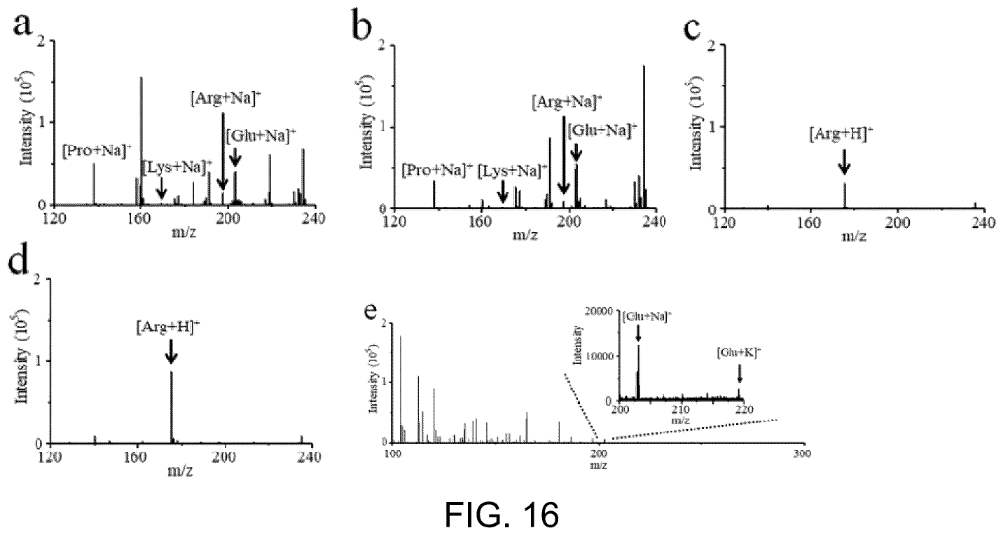

In the salt-tolerance and protein-tolerance experiment, as shown in a of FIG. 16 and b of FIG. 16, p-ZnO can still detect the mixture of the four metabolites well under high salt and high protein concentrations.

### Mass Spectrometry Detection of the Serum Metabolite

(1) Preparation of instruments and reagents: matrix-assisted laser desorption ionization mass spectrometry is set to a positive ion reflection mode for detection. The Au@ZnO nanocomposite material is prepared into a suspension for detection of standard serum.
(2) Sample preparation is performed on the mass spectrometry target plate, and drying is performed at room temperature.
(3) Detection is performed using the mass spectrometry, and the mass spectrum image is analyzed. Plasma under physiological conditions contains various inorganic salts and proteins, so the salt-tolerant and protein-resistant properties of the matrix in mass spectrometry detection has to be considered. As shown in FIG. 16, a of FIG. 16 is the mass spectrum detection result of a mixture of four metabolites (proline, lysine, arginine, and glucose are all 1 mg/mL) containing 0.2 mg/mL sodium chloride, it may be seen from the drawing that the polygonal star-shaped Au@ZnO nanocomposite material according to the embodiment can detect all four metabolites under the high salt concentration condition. In contrast to the CHCA used as a matrix in c of FIG. 16, which only detects the hydrogenation peak of arginine. Therefore, the mixture of the four metabolites (proline, lysine, arginine, and glucose are all 1 mg/mL) has good salt tolerance. b of FIG. 16 is a mixture of four metabolites (proline, lysine, arginine, and glucose are all 1 mg/mL) with 1 mg/mL bovine serum albumin added. It may be seen from b of FIG. 16 that the four metabolites can also be detected by the polygonal star-shaped Au@ZnO nanomaterial matrix in this example. In d of FIG. 16, CHCA merely detects the hydrogenation peak of arginine, so the polygonal star-shaped Au@ZnO nanocomposite material have good protein resistance properties.

It may be seen that CHCA has a poor detection effect on metabolites in high-salt and high-protein mixtures, and only the hydrogenation peak of arginine is detected, while the polygonal star-shaped Au@ZnO nanocomposite material of the disclosure can detect four metabolites, which shows that the polygonal star-shaped Au@ZnO nanocomposite material of the disclosure is more suitable for the detection of complex biological samples and has lower background interference than the conventional organic matrix CHCA.
(IV) The preparation method of the porous alloy nanomaterial use thereof in the detection of the plasma metabolite

### Example 1 Preparation of Porous Alloy Nanomaterial

Step 1: Preparation of instruments and reagents: matrix-assisted laser desorption ionization mass spectrometry is set to a positive ion reflection mode.
Step 2: Preparation of porous PdPtAu alloy including following steps
Step 2.1: 0.6 mL Na2PdCl4 (20 mM), 3mL H2PtCl6·6H2O (20 mM), 60 μL hydrochloric acid (6.0M), and 60 mg F127 are fully mixed, dissolved with an ultrasonic treatment, after F127 being completely dissolved, 3 mL of ascorbic acid solution (0.1M) is added and immediately placed in a 45° C. water bath ultrasonic treatment for 3 hours, and added 1.2 mL HAuCl4·4H2O solution (concentrations are 10 mM, 20 mM, 30 mM, and 40 mM respectively) to react for 1 hour. After the reaction, the mixture is washed three times each with absolute ethanol and water by centrifugation (10,000 rpm) and dried at 50° C. for later use.
Step 2.2: The porous PdPtAu alloy is dispersed in deionized water and used as a matrix.
Step 3: The mixture (glucose, phenylalanine, lysine) is dissolved in deionized water to detect the mass spectrometry performance of PdPtAu with different pore sizes and gold contents.
Step 4: The plasma is mixed with an equal volume of methanol/acetonitrile mixture (methanol/acetonitrile, v/v=1:1), is shaken on a shaker for 10 minutes and is centrifuged for 10 minutes, and the supernatant is taken for mass spectrometry detection.
Step 5: Sample preparation is performed on the mass spectrometry target plate, the optimized PdPtAu material is used as the matrix, and drying is performed at room temperature.
Step 6: Detection of small molecule is performed on the plasma sample.
Step 7: The mass spectrometry detection result is analyzed and conclusion is drawn.

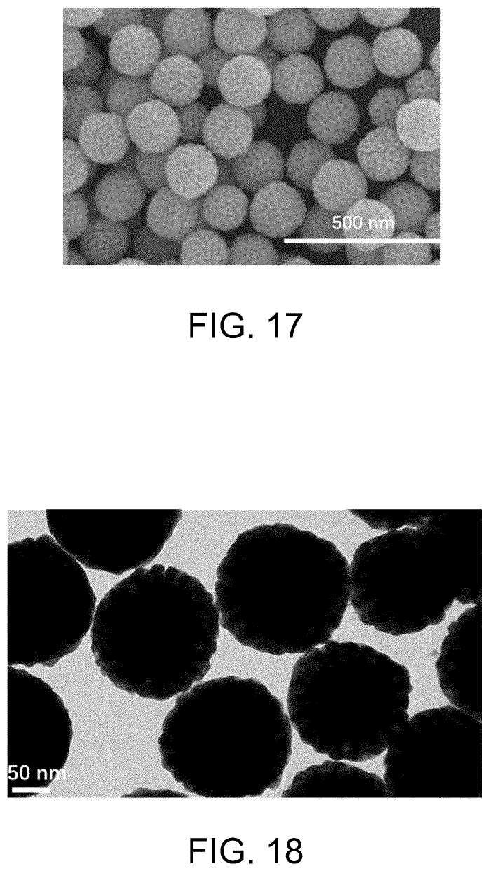

Hitachi S-4800 is used to obtain scanning electron microscopy and energy dispersive X-ray spectroscopy results. As shown in FIG. 17, the synthesized porous PdPtAu alloy has a particle size of approximately 170 nm, a uniform particle size, and a porous structure.

JEOL JEM-2100F is used to obtain a transmission electron microscopy result. As shown in FIG. 18, the nanomaterial has a porous structure, which is consistent with the scanning electron microscope result.

### Example 2 Detection of Standard Small Molecule Substance

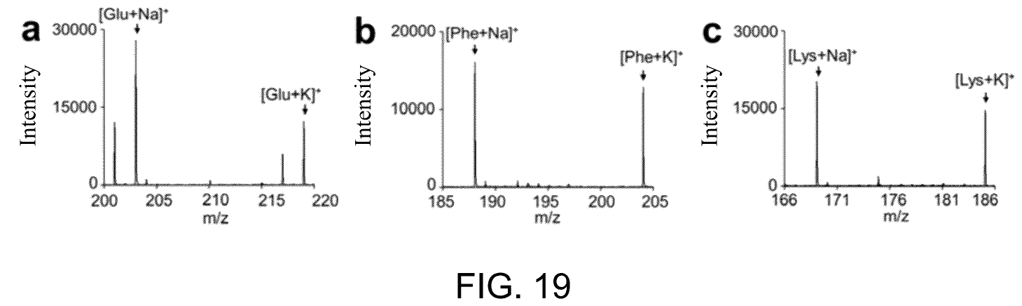

(1) Preparation of instruments and reagents: matrix-assisted laser desorption ionization mass spectrometry is set to a positive ion reflection mode for detection. The porous alloy is prepared into a suspension, and a standard small molecule (glucose, phenylalanine, and lysine) solution is prepared.
(2) Sample preparation is performed on the mass spectrometry target plate, and drying is performed at room temperature.
(3) Detection is performed using the mass spectrometry, and the mass spectrum image is analyzed. The result is shown in FIG. 19. a of FIG. 19, b of FIG. 19, and c of FIG. 19 are glucose, phenylalanine, and lysine standard small molecule solutions respectively.

### Example 3 Detection of Small Molecule Substance Containing NaCl and Bovine Serum Albumin

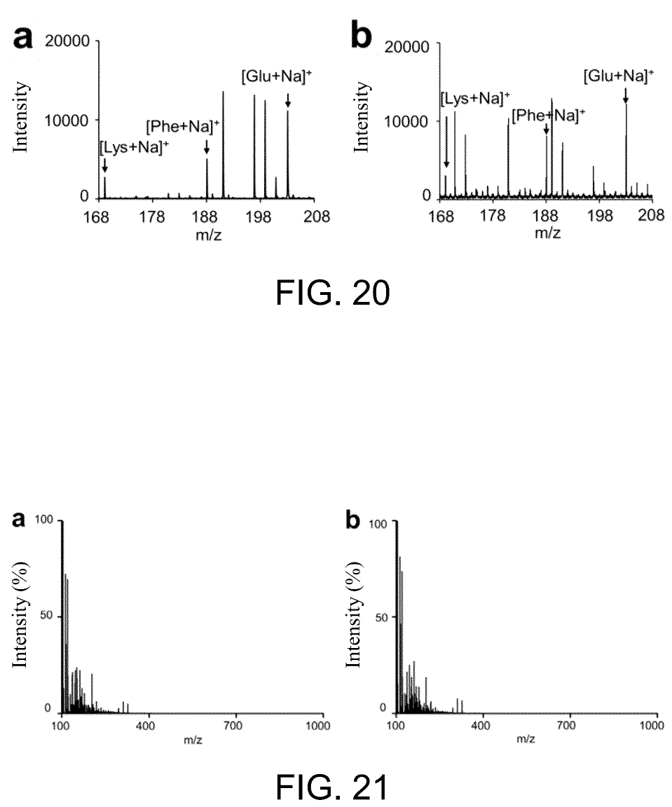

(1) Preparation of instruments and reagents: matrix-assisted laser desorption ionization mass spectrometry is set to a positive ion reflection mode for detection. The porous alloy is prepared into a suspension, and a mixture of small molecules (glucose, phenylalanine, and lysine) is prepared with high concentration of salt or protein.
(2) Sample preparation is performed on the mass spectrometry target plate, and drying is performed at room temperature.
(3) Detection is performed using the mass spectrometry, and the mass spectrum image is analyzed. The result is shown in FIG. 20. a of FIG. 20 is a mass spectrum for detecting glucose, phenylalanine, and lysine in the solution containing NaCl, and b of FIG. 20 is a mass spectrum for detecting glucose, phenylalanine, and lysine in the solution containing bovine serum albumin.

### Example 4 Detection of Plasma Metabolite

(1) Preparation of instruments and reagents: matrix-assisted laser desorption ionization mass spectrometry is set to a positive ion reflection mode for detection. The porous alloy is prepared into a suspension. The plasma is briefly pretreated with an organic solvent.
(2) Sample preparation is performed on the mass spectrometry target plate, and drying is performed at room temperature.
(3) Detection is performed using the mass spectrometry, and the mass spectrum image is analyzed. The result is shown in FIG. 21. a of FIG. 21 and b of FIG. 21 are the mass spectra of healthy plasma samples and cancer plasma samples respectively.

The above description of the embodiments is to facilitate persons of ordinary skill in the technical field to understand and use the disclosure. It should be understood that persons skilled in the art can easily make various modifications to the embodiments and apply the general principles described herein to other embodiments without inventive efforts. Therefore, the 10 disclosure is not limited to the embodiments. Based on the contents of the disclosure, improvements and modifications made by persons skilled in the art without departing from the scope of the disclosure should be within the protection scope of the disclosure.

1. A method of preparing a metal organic framework material, characterized in that the method comprises comprising steps as follows:
step 1: mixing dimethylformamide, ethanol, deionized water, triethylamine, and terephthalic acid, adding into a closed container, and adding metal chloride to form a mixture;
step 2: performing a water bath ultrasonic treatment on the mixture to obtain a reactant;
step 3: centrifuging and washing the reactant to remove residual dimethylformamide to obtain a product; and
step 4: dispersing the product in the step 3 in deionized water and separating an ultra-thin metal organic framework material by using low-speed centrifugation.

2. A method of using a metal organic framework material obtained by the method according to claim 1 in a detection of a small molecule.

3. The method according to claim 2, characterized by comprising steps as follows:
step 1: setting a detection mode of a matrix-assisted laser desorption ionization mass spectrometry to a positive ion reflection mode;
step 2: diluting a serum sample proportionally;
step 3: performing a sample preparation on a mass spectrometry target plate, using the ultra-thin metal organic framework material as a matrix, and drying at a room temperature;
step 4: performing the detection of the small molecule on the serum sample; and
step 5: analyzing an original mass spectrum and obtaining a detection result.

4. A method of preparing a sub-micron reactor, characterized by comprising steps as follows:
step 1: dissolving 3-aminophenol APF in deionized water and adding formaldehyde solution and ammonia solution;
step 2: reacting the mixture in the step 1 at 30° C. for 30 minutes; and
step 3: centrifuging and washing the reactant in the step 2 to obtain an APF submicron material.

5. A method of detecting a serum metabolite of the sub-micron reactor according to claim 4, comprising steps as follows:
step 1: preparing instruments and reagents: setting a detection mode of a matrix-assisted laser desorption ionization mass spectrometry to a positive ion reflection mode;
step 2: dispersing obtained APF-sphere&Au, APF-bowl&Au-1, APF-bowl&Au-2, and APF-bowl&Au-3 in deionized water and using as matrices;
step 3: diluting a serum sample proportionally;
step 4: performing a sample preparation on a mass spectrometry target plate, using the matrices in step 2, and drying at a room temperature;
step 5: performing a detection of a small molecule on the serum sample; and
step 6: analyzing an original mass spectrum and obtaining a detection result.

6. A method of preparing a polygonal star-shaped Au@ZnO nanocomposite material, characterized by comprising steps as follows:
step 1: preparing a nanogold solution: after stirring and heating a hydrated tetrachloroauric acid solution to 120±1° C., adding sodium citrate dihydrate, stirring at a constant temperature for 30±0.1 min, and cooling to a room temperature for later use;
step 2: preparing zinc oxide: adding a zinc acetate solution to a sodium hydroxide solution, heating to 60±1° C. and maintaining for 1±0.1 h, centrifuging to collect precipitate, washing, and drying at 50±1° C. for later use;
step 3: preparing the polygonal star-shaped Au@ZnO nanocomposite material: mixing a zinc acetate solution and the nanogold solution to obtain a mixture, adding a sodium hydroxide solution into the mixture, heating to 60±1° C. and maintaining for 1±0.1 h, centrifuging to collect precipitate, washing, and drying at 50±1° C. for later use.

7. A polygonal star-shaped Au@ZnO nanocomposite material, wherein the polygonal star-shaped Au@ZnO nanocomposite material is obtained by the preparation method according to claim 6.

8. A method of using the polygonal star-shaped Au@ZnO nanocomposite material according to claim 7 used as a matrix material in a matrix-assisted laser desorption ionization mass spectrometry detection, a matrix-assisted laser desorption ionization mass spectrometry detection of a small molecule metabolite, or a matrix-assisted laser desorption ionization mass spectrometry detection of a serum metabolite.

9. A method of preparing a porous alloy nanomaterial, comprising steps as follows:
step 1: fully mixing Na2PdCl4, H2PtCl6·6H2O, hydrochloric acid, and F127 and dissolving with an ultrasonic treatment;
step 2: after being completely dissolved, adding an ascorbic acid solution, and immediately placing in a water bath ultrasonic treatment; then
step 3: adding a HAuCl4·4H20 solution to react; and finally
step 4: centrifuging and washing with absolute ethanol and water respectively, and then drying to obtain a porous alloy nanomaterial PdPtAu.

10. A method of using a porous alloy nanomaterial obtained by the method according to claim 9 in a detection of a plasma metabolite, comprising step as follows:
step 1: dispersing the porous alloy nanomaterial PdPtAu in deionized water;
step 2: mixing plasma with an equal volume of a methanol/acetonitrile mixture, wherein a volume ratio of methanol/acetonitrile is 1:1, shaking on a shaker for 10 min and centrifuging for 10 min, and taking a supernatant for a mass spectrometry detection; and
step 3: setting a matrix-assisted laser desorption ionization mass spectrometry to a positive ion reflection mode, performing a sample preparation on a mass spectrometry target plate of the matrix-assisted laser desorption ionization mass spectrometry, wherein the sample is 1 μL of plasma extract, and performing a detection of a small molecule of a plasma sample.
